(19) United States
(12) Patent Application Publication
(10) Pub. No.: US 2020/0138794 A1
(43) Pub. Date: May 7, 2020

(54) NOVEL PARASITE THERAPY

(71) Applicant: Thomas Julius Borody, Five Dock (AU)

(72) Inventor: Thomas Julius Borody, Five Dock (AU)

(21) Appl. No.: 16/624,777

(22) PCT Filed: Aug. 15, 2018
(86) PCT No.: PCT/AU2018/000135

§ 371 Date: Dec. 19, 2019

Related U.S. Application Data
(60) Provisional application No. 62/545,989, filed on Aug. 15, 2017.

(51) Int. Cl. A61K 31/437; A61K 38/14; A61K 9/00; A61P 25/00; A61K 31/357

(52) U.S. Cl. CPC A61K 31/437; A61K 38/14; A61K 31/357; A61P 25/00; A61K 9/0056

(57) ABSTRACT

In alternative embodiments, provided are pharmaceutical compositions and methods for treating, ameliorating, reversing and/or preventing (acting as a prophylaxis) autism, e.g., regressive autism. In alternative embodiments, these pharmaceutical compositions and methods are dosaged and administered to children in need thereof. In alternative embodiments, pharmaceutical compositions and methods are dosaged, formulated and dosaged as solid, liquid or aerosol preparations or formulations. In alternative embodiments, pharmaceutical compositions comprise rifaximin as the sole antibiotic, or rixafimin and other antimicrobial or antibiotic agent, for example, vancomycin, metronidazole, tinidazole or a combination thereof.

### FIELD

This invention generally relates to medicine and gastroenterology, pharmacology and microbiology. In alternative embodiments, provided are pharmaceutical compositions and methods for treating, ameliorating, reversing and/or preventing (acting as a prophylaxis) autism or an autism spectrum disorder (ASD), e.g., regressive autism. In alternative embodiments, these pharmaceutical compositions and methods are dosaged and administered to children in need thereof. In alternative embodiments, pharmaceutical compositions and methods are dosaged, formulated and dosaged as solid, liquid or aerosol preparations or formulations. In alternative embodiments, pharmaceutical compositions comprise rifaximin as the sole antibiotic, or rixafimin and other antimicrobial or antibiotic agent, for example, vancomycin, metronidazole, tinidazole or a combination thereof.

### BACKGROUND

There are conditions which are clinically characterised by a super-infected stool, but where the bacterial super-infection can be difficult to detect these pose a medical challenge with treatment. Among such conditions are Irritable Bowel Syndrome, Colitis, and Autism/Autism Spectrum Disorder (ASD), to name a few. In ASD some researchers have identified unusual Clostridia, e.g. Clostridium boltae and Desulfovibrios. These may be an underlying pathogenic mechanism, and may cause the development and manufacture within the colonic flora of neurotoxins. Such neurotoxins can ultimately enter the peripheral circulation in a child that has acquired such an infection during a course of antibiotics in its youth—and the toxins attach themselves to various areas in the brain such as Broca's area, association areas in the parietal cortex and visual areas in the occipital cortex. When those parts of the brain are affected by the neurotoxins, neurotransmission is inhibited and the children develop the classic symptoms of Autism. This includes loss of learned speech, repetitive movements, an inability to see mother's eyes, or loss of eye contact. Given that the origin of the neurotoxins is the bowel flora—considered to be the body's largest organ albeit a ‘virtual organ’ since it does not carry human DNA cells—this super-infection can then become the target for therapy to reverse Autism. Sandler et al (Journal of Child Neurology; July 2000; 15, 7) reported that treatment of such children with vancomycin may suppress the production of these neurotoxins, resulting in reversal of behavioural changes and progress to normality within a few weeks of treatment on vancomycin, which is not absorbable. Other antibiotics can be used for this condition, for example, Rodakis reported that a child's autistic symptoms reversed almost completely when treated with an amoxycillin preparation (Rodakis J. Microbial Ecology in Health & Disease 2015; 26: 26382).

### SUMMARY OF INVENTION

In alternative embodiments, provided are compositions, including formulations, pharmaceutical compositions, foods, feeds, supplements, products of manufacture, and the like, for treating, ameliorating, reversing and/or preventing (acting as a prophylaxis) autism, e.g., regressive autism, comprising use of rifaximin as the sole antibiotic or antibacterial, or a rixafimin and another antimicrobial or antibiotic agent, for example, vancomycin, metronidazole, tinidazole or a combination thereof.

In alternative embodiments, provided are methods and compositions for treating, ameliorating, reversing and/or preventing (acting as a prophylaxis) an autism or an autism spectrum disorder (ASD), optionally a regressive autism setback-type autism, or an acquired autistic syndrome, to an individual in need thereof, comprising administering to an individual in need thereof a formulation, a pharmaceutical preparation or a pharmaceutical composition comprising or consisting of:

(a) a rifaximin (optionally a Xifaxan™, XifaxanTA™ or NORMIX™), an extended intestinal release (EIR) rifaximin, a rifamycin derivative, a rifampicin (or rifampin) (optionally RIFADIN™), a rifabutin (optionally MYCOBUTIN™), a rifapentine (optionally Priftin™), a rifalazil, a bicozamycin, or a mixture or combination thereof, or
(b) a rifaximin (optionally a Xifaxan™, XifaxanTA™ or NORMIX™) and at least one additional antimicrobial or antibiotic agent,
wherein optionally the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™) or a combination thereof.

In alternative embodiments, the autism or the autism spectrum disorder (ASD) is selected from the group consisting of autistic disorder, pervasive developmental disorder not otherwise specified (PDD-NOS), and Asperger syndrome.

In alternative embodiments, the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.

In alternative embodiments, the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentin, sulfisoxazole, sulfamethoxazole, sulfadiazine, sulfadoxine, sulfasalazine, sulfaphenazole, dapsone, sulfacytidine, linezolid or any combination thereof.

In alternative embodiments, the at least one additional antimicrobial or antibiotic agent comprises:

an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or an equivalent thereof or a combination thereof.

In alternative embodiments, the individual exhibits at least an about 5% to 10% reduction in autism or an autism spectrum disorder (ASD) symptom severity after administration of the formulation, pharmaceutical preparation or pharmaceutical composition to the individual in need thereof as compared to before initiating the administration, wherein the reduction in symptom severity is based on an assessment system selected from the group consisting of Childhood Autism Rating Scale (CARS), Childhood Autism Rating Scale 2—Standard Form (CARS2-ST), Childhood Autism Rating Scale 2—High Functioning (CARS2-HF), and a combination thereof.

In alternative embodiments, the at least about 5% to 10% reduction in autism or an autism spectrum disorder (ASD) symptom severity is achieved after about 1 to 2 or more weeks, or after about 1 to 2 months, of initiating the administration.

In alternative embodiments, the at least about 5% to 10% reduction in autism or an autism spectrum disorder (ASD) symptom severity is maintained for at least about 4 to 8 weeks after discontinuing the administration.

In alternative embodiments, the formulation, the pharmaceutical or the pharmaceutical preparation is formulated as a chewable delivery vehicle, a gum, a gummy, a candy, a lozenge, an ice cream or an ice, or a yogurt.

In alternative embodiments, a unit dosage is a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose.

In alternative embodiments, a daily dosage is about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day.

In alternative embodiments, a unit dosage is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.

In alternative embodiments, the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

In alternative embodiments, the formulation, the pharmaceutical or the pharmaceutical preparation further comprises a preservative, a benzoic acid or a potassium sorbate.

In alternative embodiments, the formulation, the pharmaceutical or the pharmaceutical preparation further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.

In alternative embodiments, the formulation, the pharmaceutical or the pharmaceutical preparation further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.

In alternative embodiments, the formulation, the pharmaceutical or the pharmaceutical preparation further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

In alternative embodiments, the formulation, the pharmaceutical or the pharmaceutical preparation further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, the formulation, the pharmaceutical or the pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

In alternative embodiments, the formulation, the pharmaceutical or the pharmaceutical preparation is contained in a delivery vehicle, product of manufacture, container, syringe, device or bag.

In alternative embodiments, the formulation, the pharmaceutical or the pharmaceutical preparation is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In a first aspect, forms of the invention described herein include the following:
1. A method for treating, ameliorating, reversing and/or preventing (acting as a prophylaxis) an autism or an autism spectrum disorder (ASD), optionally a regressive autism setback-type autism, or an acquired autistic syndrome, in an individual in need thereof, comprising administering to an individual in need thereof a formulation, a pharmaceutical preparation or a pharmaceutical composition comprising or consisting of:
   (a) a rifaximin (optionally a Xifaxan™, XifaxanTA™ or NORMIX™), an extended intestinal release (EIR) rifaximin, a rifamycin derivative, a rifampicin (or rifampin) (optionally RIFADIN™), a rifabutin (optionally MYCOBUTIN™), a rifapentine (optionally Priftin™), a rifalazil, a bicozamycin, or a mixture or combination thereof, or
   (b) a rifaximin (optionally a Xifaxan™, XifaxanTA™ or NORMIX™) and at least one additional antimicrobial or antibiotic agent,
   wherein optionally the at least one additional antimicrobial or antibiotic agent comprises a vancomycin, a metronidazole (optionally Flagyl™, Metro™), a tinidazole (optionally Fasigyn™, Simplotan™, Tindamax™) or a combination thereof
2. The method of form 1, wherein the autism or the autism spectrum disorder (ASD) is selected from the group consisting of autistic disorder, pervasive developmental disorder not otherwise specified (PDD-NOS), and Asperger syndrome.
3. The method of any one of the preceding forms of the first aspect, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
4. The method of any one of the preceding forms of the first aspect, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentin, sulfisoxazole, sulfamethoxazole, sulfadiazine, sulfadoxine, sulfasalazine, sulfaphenazole, dapsone, sulfacytidine, linezolid or any combination thereof.
5. The method of any one of the preceding forms of the first aspect, wherein the at least one additional antimicrobial or antibiotic agent comprises:
   (a) an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an anti-Clostridial agent, or a ramoplanan,
   (b) an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
   (c) a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
   (d) an equivalent thereof or a combination thereof.
6. The method of any one of the preceding forms of the first aspect, wherein the individual exhibits at least an about 5% to 10% reduction in autism or an autism spectrum disorder (ASD) symptom severity after administration of the formulation, pharmaceutical preparation or pharmaceutical composition to the individual in need thereof as compared to before initiating the administration, wherein the reduction in symptom severity is based on an assessment system selected from the group consisting of Childhood Autism Rating Scale (CARS), Childhood Autism Rating Scale 2—Standard Form (CARS2-ST), Childhood Autism Rating Scale 2—High Functioning (CARS2-HF), and a combination thereof.
7. The method of form 5, wherein the at least about 5% to 10% reduction in autism or an autism spectrum disorder (ASD) symptom severity is achieved after about 1 to 2 or more weeks, or after about 1 to 2 months, of initiating the administration.
8. The method of form 5, wherein the at least about 5% to 10% reduction in autism or an autism spectrum disorder (ASD) symptom severity is maintained for at least about 4 to 8 weeks after discontinuing the administration.
9. The method of any one of the preceding forms of the first aspect, wherein the formulation, the pharmaceutical or the pharmaceutical preparation is formulated as a chewable delivery vehicle, a gum, a gummy, a candy, a lozenge, an ice cream or an ice, or a yogurt.
10. The method of any one of the preceding forms of the first aspect, wherein a unit dosage is a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose.
11. The method of any one of the preceding forms of the first aspect, wherein a daily dosage is about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day.
12. The method of any one of the preceding forms of the first aspect, wherein a unit dosage is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
13. The method of any one of the preceding forms of the first aspect, wherein the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
14. The method of any one of the preceding forms of the first aspect, wherein the formulation, the pharmaceutical or the pharmaceutical preparation further comprises a preservative, a benzoic acid or a potassium sorbate.
15. The method of any one of the preceding forms of the first aspect, wherein the formulation, the pharmaceutical or the pharmaceutical preparation further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.
16. The method of any one of the preceding forms of the first aspect, wherein the formulation, the pharmaceutical or the pharmaceutical preparation further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
17. The method of any one of the preceding forms of the first aspect, wherein the formulation, the pharmaceutical or the pharmaceutical preparation further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
18. The method of any one of the preceding forms of the first aspect, wherein the formulation, the pharmaceutical or the pharmaceutical preparation further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-O-boswellic acid (AKBA), barley coffee components, probiotics, sinefungin, S-adenosylmethionine, S-adenosyl-homocysteine, Delisea furanones, N-sulfonyl homoserine lactones or any combination thereof.
19. The method of any one of the preceding forms of the first aspect, wherein the formulation, the pharmaceutical or the pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
20. The method of any one of the preceding forms of the first aspect, wherein the formulation, the pharmaceutical or the pharmaceutical preparation is contained in a delivery vehicle, product of manufacture, container, syringe, device or bag.
21. The method of any one of the preceding forms of the first aspect, wherein the formulation, the pharmaceutical or the pharmaceutical preparation is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In a second aspect, forms of the invention described herein include the following:
1. Use of
   (a) a rifaximin (optionally a Xifaxan™, XifaxanTA™ or NORMIX™), an extended intestinal release (EIR) rifaximin, a rifamycin derivative, a rifampicin (or rifampin) (optionally RIFADIN™), a rifabutin (optionally MYCOBUTIN™), a rifapentine (optionally Priftin™), a rifalazil, a bicozamycin, or a mixture or combination thereof, or
   (b) a rifaximin (optionally a Xifaxan™, XifaxanTA™ or NORMIX™) and at least one additional antimicrobial or antibiotic agent,
   wherein optionally the at least one additional antimicrobial or antibiotic agent comprises a vancomycin, a metronidazole (optionally Flagyl™, Metro™), a tinidazole (optionally Fasigyn™, Simplotan™, Tindamax™) or a combination thereof,
   in the manufacture of a medicament for treating, ameliorating, reversing and/or preventing (acting as a prophylaxis) an autism or an autism spectrum disorder (ASD), optionally a regressive autism setback-type autism, or an acquired autistic syndrome, in an individual in need thereof.
2. The use of form 1, wherein the autism or the autism spectrum disorder (ASD) is selected from the group consisting of autistic disorder, pervasive developmental disorder not otherwise specified (PDD-NOS), and Asperger syndrome.
3. The use of any one of the preceding forms of the second aspect, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
4. The use of any one of the preceding forms of the second aspect, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentin, sulfisoxazole, sulfamethoxazole, sulfadiazine, sulfadoxine, sulfasalazine, sulfaphenazole, dapsone, sulfacytidine, linezolid or any combination thereof.
5. The use of any one of the preceding forms of the second aspect, wherein the at least one additional antimicrobial or antibiotic agent comprises:
   (a) an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an anti-Clostridial agent, or a ramoplanan,
   (b) an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
   (c) a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
   (d) an equivalent thereof or a combination thereof
6. The use of any one of the preceding forms of the second aspect, wherein the individual exhibits at least an about 5% to 10% reduction in autism or an autism spectrum disorder (ASD) symptom severity after administration of the medicament to the individual in need thereof as compared to before initiating the administration, wherein the reduction in symptom severity is based on an assessment system selected from the group consisting of Childhood Autism Rating Scale (CARS), Childhood Autism Rating Scale 2—Standard Form (CARS2-ST), Childhood Autism Rating Scale 2—High Functioning (CARS2-HF), and a combination thereof.
7. The use of form 5, wherein the at least about 5% to 10% reduction in autism or an autism spectrum disorder (ASD) symptom severity is achieved after about 1 to 2 or more weeks, or after about 1 to 2 months, of initiating administration of the medicament.
8. The use of form 5, wherein the at least about 5% to 10% reduction in autism or an autism spectrum disorder (ASD) symptom severity is maintained for at least about 4 to 8 weeks after discontinuing administration of the medicament.
9. The use of any one of the preceding forms of the second aspect, wherein the medicament is formulated as a chewable delivery vehicle, a gum, a gummy, a candy, a lozenge, an ice cream or an ice, or a yogurt.
10. The use of any one of the preceding forms of the second aspect, wherein the medicament is formulated as a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose.
11. The use of any one of the preceding forms of the second aspect, wherein the medicament is formulated for a daily dosage of about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day.
12. The use of any one of the preceding forms of the second aspect, wherein the medicament is formulated for administration bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
13. The use of any one of the preceding forms of the second aspect, wherein the medicament further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
14. The use of any one of the preceding forms of the second aspect, wherein the medicament further comprises a preservative, a benzoic acid or a potassium sorbate.
15. The use of any one of the preceding forms of the second aspect, wherein the medicament further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.
16. The use of any one of the preceding forms of the second aspect, wherein the medicament further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
17. The use of any one of the preceding forms of the second aspect, wherein the medicament further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
18. The use of any one of the preceding forms of the second aspect, wherein the medicament further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
19. The use of any one of the preceding forms of the second aspect, wherein the medicament is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
20. The use of any one of the preceding forms of the second aspect, wherein the medicament is contained in a delivery vehicle, product of manufacture, container, syringe, device or bag.
21. The use of any one of the preceding forms of the second aspect, wherein the medicament is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In a third aspect, forms of the invention described herein include the following:
1. A composition comprising or consisting of:
   (a) a rifaximin (optionally a Xifaxan™, XifaxanTA™ or NORMIX™), an extended intestinal release (EIR) rifaximin, a rifamycin derivative, a rifampicin (or rifampin) (optionally RIFADIN™), a rifabutin (optionally MYCOBUTIN™), a rifapentine (optionally Priftin™), a rifalazil, a bicozamycin, or a mixture or combination thereof, or
   (b) a rifaximin (optionally a Xifaxan™, XifaxanTA™ or NORMIX™) and at least one additional antimicrobial or antibiotic agent,
   wherein optionally the at least one additional antimicrobial or antibiotic agent comprises a vancomycin, a metronidazole (optionally Flagyl™, Metro™), a tinidazole (optionally Fasigyn™, Simplotan™, Tindamax™) or a combination thereof,
   for use in treating, ameliorating, reversing and/or preventing (acting as a prophylaxis) an autism or an autism spectrum disorder (ASD), optionally a regressive autism setback-type autism, or an acquired autistic syndrome, in an individual in need thereof
2. The composition of form 1, wherein the autism or the autism spectrum disorder (ASD) is selected from the group consisting of autistic disorder, pervasive developmental disorder not otherwise specified (PDD-NOS), and Asperger syndrome.
3. The composition of any one of the preceding forms of the third aspect, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
4. The composition of any one of the preceding forms of the third aspect, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentin, sulfisoxazole, sulfamethoxazole, sulfadiazine, sulfadoxine, sulfasalazine, sulfaphenazole, dapsone, sulfacytidine, linezolid or any combination thereof.
5. The composition of any one of the preceding forms of the third aspect, wherein the at least one additional antimicrobial or antibiotic agent comprises:
   (a) an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an anti-Clostridial agent, or a ramoplanan,
   (b) an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
   (c) a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
   (d) an equivalent thereof or a combination thereof.
6. The composition of any one of the preceding forms of the third aspect, wherein the individual exhibits at least an about 5% to 10% reduction in autism or an autism spectrum disorder (ASD) symptom severity after administration of the composition to the individual in need thereof as compared to before initiating the administration, wherein the reduction in symptom severity is based on an assessment system selected from the group consisting of Childhood Autism Rating Scale (CARS), Childhood Autism Rating Scale 2—Standard Form (CARS2-ST), Childhood Autism Rating Scale 2—High Functioning (CARS2-HF), and a combination thereof.
7. The composition of form 5, wherein the at least about 5% to 10% reduction in autism or an autism spectrum disorder (ASD) symptom severity is achieved after about 1 to 2 or more weeks, or after about 1 to 2 months, of initiating administration of the composition.
8. The composition of form 5, wherein the at least about 5% to 10% reduction in autism or an autism spectrum disorder (ASD) symptom severity is maintained for at least about 4 to 8 weeks after discontinuing administration of the composition.
9. The composition of any one of the preceding forms of the third aspect, wherein the composition is formulated as a chewable delivery vehicle, a gum, a gummy, a candy, a lozenge, an ice cream or an ice, or a yogurt.
10. The composition of any one of the preceding forms of the third aspect, wherein a unit dosage of the composition is a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose.
11. The composition of any one of the preceding forms of the third aspect, wherein a daily dosage of the composition is about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day.
12. The composition of any one of the preceding forms of the third aspect, wherein a unit dosage of the composition is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
13. The composition of any one of the preceding forms of the third aspect, further comprising a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
14. The composition of any one of the preceding forms of the third aspect, further comprising a preservative, a benzoic acid or a potassium sorbate.
15. The composition of any one of the preceding forms of the third aspect, further comprising: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.
16. The composition of any one of the preceding forms of the third aspect, further comprising: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
17. The composition of any one of the preceding forms of the third aspect, further comprising an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
18. The composition of any one of the preceding forms of the third aspect, further comprising: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
19. The composition of any one of the preceding forms of the third aspect, wherein the composition is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
20. The composition of any one of the preceding forms of the third aspect, wherein the composition is contained in a delivery vehicle, product of manufacture, container, syringe, device or bag.
21. The composition of any one of the preceding forms of the third aspect, wherein the composition is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

The details of one or more embodiments of the invention are set forth in the accompa-nying description below. Other features, objects, and advantages of the invention will be apparent from the description and the claims.

All publications, patents, patent applications cited herein are hereby expressly incorporated by reference for all purposes.

### DESCRIPTION OF EMBODIMENTS

In alternative embodiments, provided are pharmaceutical compositions and methods for treating, ameliorating, reversing and/or preventing (acting as a prophylaxis) autism or an autism spectrum disorder (ASD), e.g., regressive autism. In alternative embodiments, these pharmaceutical compositions and methods are dosaged and administered to children in need thereof. In alternative embodiments, pharmaceutical compositions and methods are dosaged, formulated and dosaged as solid, liquid or aerosol preparations or formulations. In alternative embodiments, pharmaceutical compositions comprise rifaximin as the sole antibiotic, or rixafimin and other antimicrobial or antibiotic agent, for example, vancomycin, metronidazole, tinidazole or a combination thereof.

In alternative embodiments, antibiotics and antibacterials used to practice methods as provided herein are formulated and dosaged for oral administration as a powder, e.g., a lyophilised powder, which can be inserted into carriers, e.g., capsules, tablets, geltabs, and the like, e.g., for administration to autistic infants or children (or those suspected of developing autism) to ingest.

Because autism may present itself at the age of 2.5 years or above the children are unlikely to be able to swallow a capsule; thus, this provided are additional delivery vehicles, products of manufacture and devices to be combined with formulations as provided herein, e.g., powders such as lyophilised powders, e.g., lyophilised powder in a storage vehicle, e.g., capsules, geltabs and the like; for example, provided are delivery vehicles, products of manufacture and devices manufactured as a container, a kit, a package or a pack of a “device and capsule” together, e.g., operably associated such that the container, kit, package or a pack permits individuals, e.g., the very young children and the older children (and including disabled or handicapped individuals) to ingest the product, e.g., the lyophilised product, from the storage vehicle, e.g., capsules, geltabs and the like.

In alternative embodiments, the container, kit, a package or a pack provides the ability of any age child (or disabled or handicapped individual) to ingest or swallow the product within the storage vehicle (e.g., capsule) by “draining”, e.g., by puncturing, crushing or otherwise opening, the storage vehicle using a puncturing, crushing or equivalent device (operably built into the container, kit, package or pack), and allowing passage or contact of the contents of the storage vehicle to an ingestible liquid, which is also contained within the container, kit, package or pack, which can be initially (before the puncturing, crushing or otherwise opening) in a separate compartment from the storage compartment. This puncturing, crushing or otherwise opening of the storage compartment and the passage or contact of the contents of the storage vehicle to the ingestible liquid effectively places the contents of the storage, e.g., a powder, into the ingestible liquid, which can be e.g., water, a milk, a yoghurt, an ice cream, a yogurt, a juice (e.g., a fruit juice, an apple juice) or a masking drink. The container, kit, package or pack can be designed as an infant feeding bottle, e.g., comprising a nipple or teat for the very young.

In alternative embodiments, this simple puncturing or crushing device allows the storage containers, e.g., geltabs or capsules, to be punctured and/or crushed or otherwise “opened”, allowing the contents of the storage container, e.g., the powder, to fall out in to the liquid compartment, e.g., to the bottom end of a device or straight into a bottle or a container held underneath or configured to be attached and underneath. For example, in this way a provider, e.g., the mother, can purchase a supply of storage containers, e.g., geltabs or capsules, convert them as needed into a powder capable of being mixed a liquid of her choice that the child will be ingesting.

In alternative embodiments, for those capable of swallowing tablets, capsules and the like, the storage containers, e.g., geltabs, tablets or capsules, are manufactured as enteric coated to bypass the acid of the stomach and bile of the duodenum, such that the storage containers, e.g., geltabs, tablets or capsules open (e.g., dissolve) in the jejunum or below.

In alternative embodiments, further provided are instructions for use, e.g., that when emptied into a drink, providers (e.g., the mothers of infants or children) are advised to choose a drink that has its own buffering capacity such as flavoured milk, chocolate milk, ice cream, yoghurt, ice blocks, frozen icicles, or simply milk, e.g., that is being fed to the infant or child by a bottle, e.g., a milk bottle, with a nipple or teat.

In alternative embodiments, storage containers, e.g., geltabs, tablets or capsules, or any formulation as provided herein, also comprises an antacid, e.g., a calcium carbonate, magnesium hydroxide, propylene glycol alginate and sodium alginate, or the combination of aluminium hydroxide with magnesium trisilicate, magnesium oxide or magnesium carbonate, so that when the storage container is punctured, crushed or otherwise opened and put into contact with the liquid, e.g., the feeding bottle, and ingested, there will be greater protection from acid damage. In alternative embodiments, methods and instructions further comprise the infant or child also being given an acid suppressant beforehand to permit more viable living bacteria to arrive in the colon.

In alternative embodiments, formulations, pharmaceuticals or pharmaceutical preparations as provided herein are formulated or manufactured as storage vehicles, e.g., tablets, geltabs, pills, capsules and the like; and in alternative embodiments, these storage vehicles are contained in, or contained in a kit with, or packaged with, or sold together with, a storage vehicle ‘cracking’, puncturing, or otherwise opening or releasing device (, e.g., as a powder, e.g., as lyophilised material). These can be dispensed together, or configured together, or manufactured together, as a simple way of meeting the needs of both infants, the very young, older children and needful (e.g., handicapped) adults; e.g., as a powder, e.g., as lyophilised material, e.g., from their storage vehicles, e.g., as encapsulated formulations, pharmaceuticals or pharmaceutical preparations, thus permitting successful clinical administration on a frequent, e.g., bid, tid, or daily, basis for prolonged periods.

### Methods of Use and Applications of Devices and Compositions

In alternative embodiments, provided are compositions, devices and methods for treating, ameliorating, reversing and/or preventing (acting as a prophylaxis) autisms, including the so-called regressive autism, autism with regression, autistic regression, setback-type autism, or acquired autistic syndrome. In alternative embodiments, provided are compositions, devices and methods for treating, ameliorating, reversing and/or preventing (acting as a prophylaxis): Autism Spectrum Disorder, including Autism itself, Asperger's Syndrome, Pervasive Developmental Disorder, and Childhood Disintegrative Disorder. In alternative embodiments, provided are compositions and methods for treating, ameliorating, reversing and/or preventing (acting as a prophylaxis) Rett Syndrome, Obsessive Compulsive Disorders (OCD), various anxiety disorders as well as major depressive disorders, bipolar disorders, anorexia nervosa, bulimia nervosa, Tourette's syndrome, Attention Deficit Hyperactivity Disorder, Trichotillomania and Dermatillomania.

### Multicomponent Packaging

Provided are multi-component delivery systems, e.g., products of manufacture, comprising e.g., formulations, pharmaceutical preparations or pharmaceutical compositions used to practice methods as provided herein, e.g., formulated and dosaged for oral administration as a powder, e.g., a lyophilised powder, and another component, e.g., a liquid; these multi-component delivery systems, e.g., products of manufacture, can be designed or manufactured as described e.g., in U.S. Pat. Nos. 8,968,717; 8,931,665; 7,861,854; 7,018,089; 6,626,912; and, U.S. Pat. App. Pub nos. 2010/0034574; 2009/0180923; 20090232886; 2008/0160076; 2007/0087048; 2007/0036830; 2007/0074979; 2005/0205438; 2004/0089563.

### Packaging

Provided are compositions, including preparations, formulations and/or kits, comprising combinations of ingredients, as described herein. In alternative embodiments, these combinations can be mixed and administered together, or alternatively, they can be an individual member of a packaged combination of ingredients, e.g., a liquid component and a solid product component manufactured in a separate compartment, package, kit or container; e.g., where all or a subset of the combinations of ingredients are manufactured in a separate compartment, package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a formulations, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of formulations, pharmaceutical preparations or pharmaceutical compositions as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

In alternative embodiments, formulations, pharmaceutical preparations or pharmaceutical compositions are formulated, e.g., as a powder, e.g., as lyophilised material, e.g., a lyophilized encapsulated product, e.g., for practicing methods as provided herein, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of formulations, pharmaceutical preparations or pharmaceutical compositions as provided herein. Products or kits comprise an aqueous solution(s) which are dispensed (e.g., by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, products of manufacture include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic agent. This safeguards the therapeutic agent's efficacy by protecting each tablet, geltab or pill until it's taken; gives the product or kit portability, makes it easy to take a dose anytime or anywhere.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

A 14 y old autistic child (averbal) with OCD and severe abdominal pains, gas and constipation, was commenced on Rifaximin 500 mg bid, metronidazole 200 bid later increased to 400 bid, to treat the gastrointestinal symptoms. Progressively his bowel symptoms improved, albeit incompletely and vancomycin 250 mg bid was added. Over the next 6 months his abdominal symptoms completely resolved and he could sleep through the night without being woken by pains. At about 9 months on continuous treatment the parents reported on a Skype consultation in the presence of his two carers that the boy now went to school, could speak more than 800 words, and carried out commands. During the consultation, the child was asked to go to the garage and bring back his father's coat which was on the back seat of the car. The child promptly carried out the request. He continues to be on the oral drugs and attends school.

### Example 2

A 22 y old male patient with ASD unable to say more than 2 word-like noises (mamma and ‘no’) was brought for treatment mainly because of his aggressive behaviour (requiring two carers holding him in the office) and self-damaging OCD. He hit his head against a brick wall till his scalp was deeply cut to visible bone, and bleeding. He was placed on tinidazole 500 mg bid, rifaximin 500 mg bid increasing to 1 g bid after 2 weeks, and vancomycin 250 mg ii bid. Over the next 6 months the patient had progressively lost his aggression, could sit and watch movies unattended, and had progressively lost most of his OCD symptoms—especially that of chewing a large rubber ‘snake’ toy. Over the next 6 months the patient learned to speak 3 words. He continues on the medications given the marked improvement in his behaviour and word growth.

1. A method for treating, ameliorating, reversing and/or preventing (acting as a prophylaxis) an autism or an autism spectrum disorder (ASD), optionally a regressive autism setback-type autism, or an acquired autistic syndrome, in an individual in need thereof, comprising administering to the individual in need thereof a formulation, a pharmaceutical preparation or a pharmaceutical composition comprising or consisting of:
(a) a rifaximin (optionally a XIFAXAN™, XIFAXANTA™ or NORMIX™), an extended intestinal release (EIR) rifaximin, a rifamycin derivative, a rifampicin (or rifampin) (optionally RIFADIN™), a rifabutin (optionally MYCOBUTIN™), a rifapentine (optionally PRIFTIN™), a rifalazil, a bicozamycin, or a mixture or combination thereof, or
(b) a rifaximin (optionally a Xifaxan™, XifaxanTA™ or NORMIX™) and at least one additional antimicrobial or antibiotic agent,
wherein optionally the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™) or a combination thereof.

2. The method of claim 1, wherein the autism or the autism spectrum disorder (ASD) is selected from the group consisting of autistic disorder, pervasive developmental disorder not otherwise specified (PDD-NOS), and Asperger syndrome.

3. The method of any one of the preceding claims, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.

4. The method of any one of the preceding claims, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentin, sulfisoxazole, sulfamethoxazole, sulfadiazine, sulfadoxine, sulfasalazine, sulfaphenazole, dapsone, sulfacytidine, linezolid or any combination thereof.

5. The method of any one of the preceding claims, wherein the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.

6. The method of any one of the preceding claims, wherein the individual exhibits at least an about 5% to 10% reduction in autism or an autism spectrum disorder (ASD) symptom severity after administration of the formulation, pharmaceutical preparation or pharmaceutical composition to the individual in need thereof as compared to before initiating the administration, wherein the reduction in symptom severity is based on an assessment system selected from the group consisting of Childhood Autism Rating Scale (CARS), Childhood Autism Rating Scale 2—Standard Form (CARS2-ST), Childhood Autism Rating Scale 2—High Functioning (CARS2-HF), and a combination thereof.

7. The method of claim 5, wherein the at least about 5% to 10% reduction in autism or an autism spectrum disorder (ASD) symptom severity is achieved after about 1 to 2 or more weeks, or after about 1 to 2 months, of initiating the administration.

8. The method of claim 5, wherein the at least about 5% to 10% reduction in autism or an autism spectrum disorder (ASD) symptom severity is maintained for at least about 4 to 8 weeks after discontinuing the administration.

9. The method of any one of the preceding claims, wherein the formulation, the pharmaceutical or the pharmaceutical preparation is formulated as a chewable delivery vehicle, a gum, a gummy, a candy, a lozenge, an ice cream or an ice, or a yogurt.

10. The method of any one of the preceding claims, wherein a unit dosage is a pediatric unit dosage, and optionally the unit dosage is between about 10 mg and 1100 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000 or 1100 or more mg per unit dose.

11. The method of any one of the preceding claims, wherein a daily dosage is about 100, 125, 150, 175, 200, 225, 250, 275, 300, 325, 350, 375, 400, 425, 450, 475, 500, 600, 700, 750, 800, 900, 1000 or 1100 or more mg per day, or between about 100 and 1100 mgm per day.

12. The method of any one of the preceding claims, wherein a unit dosage is set for bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.

13. The method of any one of the preceding claims, wherein the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

14. The method of any one of the preceding claims, wherein the formulation, the pharmaceutical or the pharmaceutical preparation further comprises a preservative, a benzoic acid or a potassium sorbate.

15. The method of any one of the preceding claims, wherein the formulation, the pharmaceutical or the pharmaceutical preparation further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an E coli, a Strep fecalis and equivalents.

16. The method of any one of the preceding claims, wherein the formulation, the pharmaceutical or the pharmaceutical preparation further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.

17. The method of any one of the preceding claims, wherein the formulation, the pharmaceutical or the pharmaceutical preparation further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

18. The method of any one of the preceding claims, wherein the formulation, the pharmaceutical or the pharmaceutical preparation further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

19. The method of any one of the preceding claims, wherein the formulation, the pharmaceutical or the pharmaceutical preparation is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

20. The method of any one of the preceding claims, wherein the formulation, the pharmaceutical or the pharmaceutical preparation is contained in a delivery vehicle, product of manufacture, container, syringe, device or bag.

21. The method of any one of the preceding claims, wherein the formulation, the pharmaceutical or the pharmaceutical preparation is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.
